SEQUENCE LISTING (2 sequences)

SEQ ID NO: 1 - DNA, 38 bp, Homo sapiens
  source 1..38: /mol_type=“genomic DNA” /organism=“Homo sapiens”
cccctcctccctcagacccaggaggccaggccccccag

SEQ ID NO: 2 - DNA, 37 bp, Homo sapiens
  source 1..37: /mol_type=“genomic DNA” /organism=“Homo sapiens”
cccctcctccctcagacccaggaggccaggcccccag

(19) United States
(12) Patent Application Publication
(10) Pub. No.: US 2015/0361497 A1
(43) Pub. Date: Dec. 17, 2015

(54) THERAPEUTICS AND DIAGNOSTICS BASED ON MINISATELLITE REPEAT ELEMENT 1 (MSR1)

(71) Applicant: Anna Mary ROSE, London (GB)

(72) Inventor: Anna Mary ROSE, London (GB)

(21) Appl. No.: 14/761,952

(22) PCT Filed: Jan. 15, 2014
(86) PCT No.: PCT/GB2014/050107

§ 371 Date: Jul. 20, 2015

(30) Foreign Application Priority Data
Jan. 18, 2013 (GB) 1300932.9
Jan. 18, 2013 (GB) 1300944.4

(51) Int. Cl. C12Q 1/68

(52) U.S. Cl. CPC C12Q 1/6883; C12Q 2600/118; C12Q 2600/158; C12Q 2600/106

(57) ABSTRACT

The use of minisatellite repeat element 1 (MSR1) in a process of identifying therapeutic agents for use in the treatment or therapy of diseases or conditions relating to one or more genes associated with MSR1 or functional variants or derivatives thereof or use in a process of gene therapy. Also provided are tests for the prediction, diagnosis, prognosis or response to therapy in a disease or condition in a subject, said disease or condition relating to one or more genes associated with a minisatellite repeat element 1 (MSR1) or functional variants or derivatives thereof wherein said test is or comprises means for assessing the copy number variation (CNV) at an MSR1 locus of the gene or genes so as to determine the risk of the disease or condition being present or developing in the subject. Also provided are processes and kits for said tests, and targeted screening or genotyping programs using said tests.

### FIELD OF INVENTION

The present invention relates to genetic sequences, genetic sequence analyses and their use and analysis in research and development, especially in addressing areas of unmet medical need. In particular the present invention relates to the use of a specific minisatellite repeat element termed MSR1 in diagnosis, therapy and identification of new treatments for disease.

### BACKGROUND OF INVENTION

In 1987, a minisatellite repeat element was identified as a 37 bp repetitive intronic sequence in the ApoCII gene, and this sequence and its variants were later termed MSR1 [1, 2]. MSR1 elements were subsequently described at several loci, specifically, the kallikrein gene cluster, TNNI3 and PRPF31 in the human genome, and PRSS17 in the mouse genome [3-9]. PRPF31 encodes the ubiquitous splicing factor PRPF31 which has been implicated in the retinal disease autosomal dominant retinitis pigmentosa (adRP) which leads to blindness. In the human Kallikrein gene cluster ten clusters of MSR1 elements were found to be distributed along the locus. While some differences between the frequency of alleles were seen between a small sample of cancer and control patients no functional role for the element was suggested.

The element has been described as being chromosome 19 specific, with predominance at chromosome 19q13.2-13.4 [1-3] but without functional work assigning any specific role of the element beyond as markers. It has been suggested that the elements might play a role in the TNNI3 promoter [7] or in mediating formation of cis sense-antisense chimeric transcripts in prostatic cancer cells [10].

In a study of an MSR1 element cluster (hg19 co-ordinates, chr19:54618105-54618472) laying in close proximity to the PRPF31 core promoter the element was present in either three or four copies. It was demonstrated that, in isolation, copy number variation (CNV) of the MSR1 element had a modest effect on luciferase reporter assay, with the 3-copy reporter construct having 2.3-4.5 times higher activity than the 4-copy construct [8].

Retinitis pigmentosa (RP) is a genetically heterogeneous group of disorders characterised by progressive degeneration of the retinal photoreceptors (the rod and cone cells). The disease affects approximately 1/3000 individuals worldwide and is characterised initially by night blindness, followed by a constriction of visual fields and, finally, loss of central visual acuity. The disease can be caused by all manner of Mendelian inheritance, including autosomal recessive, dominant and X-linked forms. Autosomal dominant retinitis pigmentosa (adRP) accounts for 30-40% of cases; one form of adRP is associated with mutations in the ubiquitous splicing factor PRPF31.

A major adRP locus, termed RP11, was identified at chromosome 19q13.4; subsequently, PRPF31 was found to be the causative gene underlying this linkage [11, 12]. All manner of mutations have been identified, including nonsense, missense, insertions and deletions; cumulatively, PRPF31 mutations account for 5% of adRP and is the second most frequent cause of dominant disease [13-14]. A key feature of PRPF31-associated adRP is phenotypic non-penetrance, whereby there are entirely asymptomatic mutation carriers. This is due to differential expression of PRPF31 in the population: co-inheritance of a mutant allele and a low-expressing wildtype allele results in disease, whereas co-inheritance of a mutant and a high-expressing allele prevents clinical manifestation. It has been demonstrated that expression of the wildtype allele is over two-fold higher in asymptomatic individuals compared to their symptomatic relatives [15]. Differential expression of PRFP31 has been demonstrated in the normal population, and follows an almost continuous distribution, suggesting the possibility of polygenic control of gene expression [16]. It has been demonstrated that the major locus determining non-penetrance lies on the wildtype chromosome 19q13.4, in close proximity to PRPF31 [17,18]. It was, therefore, considered most likely that a factor acting in cis-relative to PRPF31 was the major factor controlling PRPF31 expression level in the population.

At present, although diagnostic testing can determine whether an individual is a PRPF31 mutation carrier, there is no test available to determine whether that individual will be symptomatic or asymptomatic. This makes genetic counselling in PRPF31-associated adRP problematic.

Cancer and associated diseases remain an area of serious unmet medical need despite major advances in research, especially in genetics, and the development of treatments and diagnostic methods. Carcinoma of the ovaries is the second most common gynaecological cancer and the sixth most common cancer in women in developed countries.

Ovarian cancer in particular has a very poor prognosis, which has been attributed to the unavailability of effective screening tools, the absence of clinical symptoms in many patients, and presentation at advanced stage of disease. A major barrier to development of effective screening and treatment is the poor understanding of pathogenesis and histiogenesis of the disease. Besides monogenic mutations in familial cancer syndromes, there has been little progress in the identification of somatic genetic changes that predispose an individual to the development of ovarian cancer, the only convincing example being variants in BNC2 [19].

One study demonstrated that PRPF31 haplotypes were associated with risk of invasive disease (p=0.03), but in depth analysis failed to show significant association with any individual SNP, suggesting that an ungenotyped variant in close proximity to the PRPF31 haplotype is responsible [20]. Increased expression of PRPF31 predicted response to chemotherapy and disease early relapse among women with advanced-stage high-grade epithelial ovarian cancer [21].

The promoter region of PRPF31 has been characterized as the genomic fragment spanning −397 to +539 relative to the annotated PRPF31 transcription start site, this fragment being termed BiP [8]. An element observed lying closely upstream of the promoter element (hg19 co-ordinates, chr19:54618105-54618472) was found to be present in the normal population copy in either three or four copies [8, Supplemental data].

Breast cancer is the most common cancer affecting women developed countries, with a 7.1% lifetime risk of developing disease. Many gene loci have been implicated in the risk of developing breast cancer, including several genes at the kallikrein locus. A cluster of MSR1 elements lays within the 3′UTR of the KLK14 genes (chr19:51580818-51581230), and it has been reported that the 9-copy allele was significantly more frequent in a small sample of patients with histologically confirmed breast cancer compared to matched control individuals [22]. Furthermore, KLK14 expression is dysregulated in several cancers, including breast, ovarian, prostate and testicular tumours [23].

Prostate cancer is the most frequent cancer in men, with more than 80% of men developing the disease by age 80. It is the sixth most common cause of cancer deaths in the developed world. In a very small number of samples, a cluster of MSR1 elements at KLK4 (chr19:51409713-51410118) showed allele heterogeneity in normal and cancerous prostate cells; furthermore, the same MSR1 element was found to be important for the formation of cis sense-antisense transcripts in cancerous cells [3, 10].

### SUMMARY OF THE INVENTION

The present invention is based on the finding of a correlation between the copy number variation (CNV) in MSR1 elements in populations and possession and/or predisposition and/or prognosis of particular biological conditions and the therapeutic manipulation of MSR1 elements.

Accordingly the present invention provides the use of minisatellite repeat element 1 (MSR1) in a process of identifying therapeutic agents or as a target, for use in the treatment or therapy of diseases or conditions relating to one or more genes associated with a minisatellite repeat element 1 (MSR1) or functional variants or derivatives thereof.

MSR1 and functional variants or derivatives thereof would be understood to comprise the elements represented in FIG. 1 and variants thereof and includes for example the prototypic sequences as shown in FIG. 1 and below:

CCCCTCCTCCCTCAGACCCAGGAGGCCAGGCCCCCCAG
CCCCTCCTCCCTCAGACCCAGGAGGCCAGGCCCCCAG

Also included are any other sequences and variants thereof defined by searching on standard repeat element databases such as RepeatMasker (www.repeatmasker.org) and RepBase (www.girinst.org/repbase/).

In a further aspect the present invention provides the use of minisatellite repeat element 1 (MSR1), or functional variants or derivatives thereof, in a process of gene therapy.

In a further aspect the present invention provides a test for the prediction, diagnosis, prognosis or response to therapy in a disease or condition in a subject, said disease or condition relating to one or more genes associated with a minisatellite repeat element 1 (MSR1) or functional variants or derivatives thereof wherein said test is or comprises means for assessing the copy number variation (CNV) at an MSR1 locus of the gene or genes so as to determine the risk of the disease or condition being present or developing in the subject.

In a further aspect the present invention provides a kit for carrying out the test of the invention. In a further aspect the present invention provides a targeted screening program using the test or method of the invention.

Embodiments of the invention include tests, kits, methods and screening programs wherein the gene is selected from one or more cancer genes, for example selected from those listed in Tables 2 or 3. The test can be PCR based.

In one aspect of the invention the present invention provides tests to assess the CNV of the MSR1 element at PRPF31 locus based on an association of the CNV of MSR1 element at the PRPF31 locus as being responsible for altered gene expression of PRPF31 in ovarian cancer patients and the observed association between PRPF31 haplotypes and ovarian carcinoma.

In one embodiment there is provided a PCR based system that used one fluorescently labelled primer and sized the PCR product against a standard ladder, thereby allowing definition of genotype.

In a further aspect of the invention there is provided a screening program to allow identification of individuals who are at increased risk, in particular, of developing ovarian carcinoma and, therefore, require regular follow-up to detect disease at an earlier stage. Furthermore since differential regulation of some MSR1 containing genes has been associated with response to chemotherapeutic agent therapy (e.g. PPP1R15A and mesothelioma [24]; KLK3 in breast cancer [25]), should CNV of MSR1 be found to underlie this differential regulation of expression, ascertainment of genotype would allow prediction of response to chemotherapeutic agents, thereby allowing cytotoxic therapy to be initiated in those most likely to respond and not in those in whom therapy is likely to be unsuccessful.

There now follow non-limiting examples and figures illustrating the invention.

### BRIEF DESCRIPTION OF THE FIGURES

FIG. 1 shows a positional weight matrix of the consensus MSR1 sequence. Although the exact sequence of individual MSR1 elements varies, it is possible to describe a consensus, or prototypic, sequence. Here, 100 individuals MSR1 sequences were selected and a positional weight matrix generated, to show which bases are most highly conserved. The height of the letter is proportional to the level of conservation, and variations are seen as smaller letters underneath the main base. The base marked with an asterisk is frequently absent, hence the often quoted 37-38 bp length.

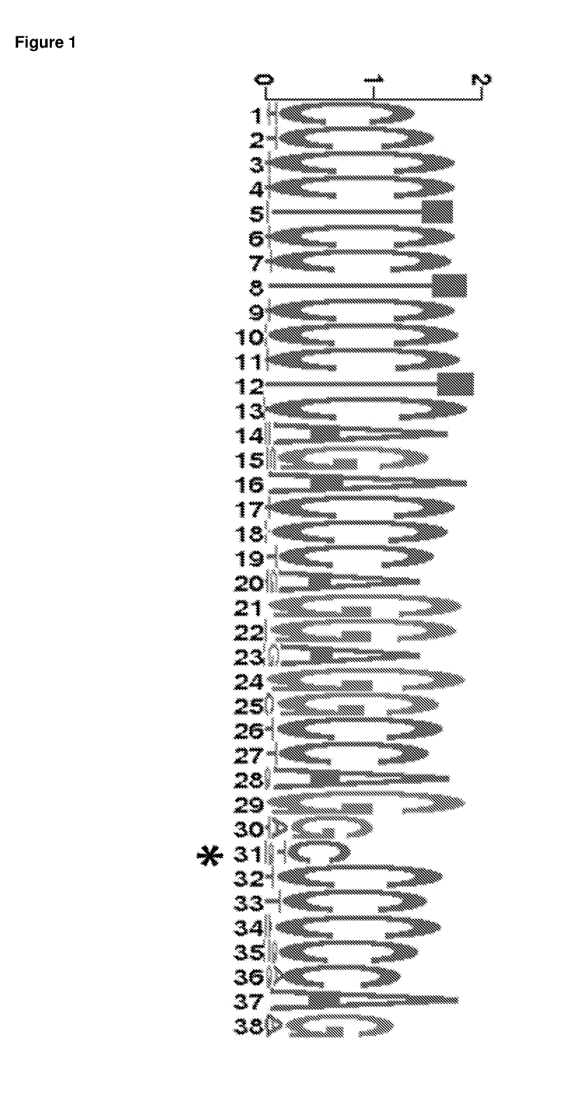

FIG. 2. shows—A: Schematic representation of the regions tested by dual-luciferase reporter assay, showing the original core promoter fragment (BiP), a fragment that showed significant differences in reporter activity in the initial study [8] and the fragment comprising both of these (BiP-SNP). B: Results of dual luciferase reporter assay in HeLa cells. C: Results of dual luciferase reporter assay in RPE-1 cells.

FIG. 3 shows A, B: Schematic representation of reporter constructs tests. A: four constructs, with variable MSR1 sequence cloned immediately upstream to pTK. B: Illustration of genomic position of MSR1 elements in two individuals (RP15011, 111.7). C, D: Results of dual-luciferase reporter assay in forwards strand (C) and reverse strand (D) directions (HeLa cell line).

FIG. 4 shows—Genotype frequency (A) and allele frequency (B) of copy number variants of MSR1 element cluster in the 3′UTR of KLK14, as ascertained in 180 control individuals (L—11 copy allele, M—9 copy allele, S—8 copy allele). C: Results of dual luciferase reporter assay in RPE-1 cell line, demonstrating a functional effect of CNV on reporter activity, regardless of element orientation.

### DETAILED DESCRIPTION AND EXEMPLIFICATION OF THE INVENTION

It will be evident to those skilled in the art that the scope of the invention is not limited by the examples and modifications are possible without departing from the scope of the invention.

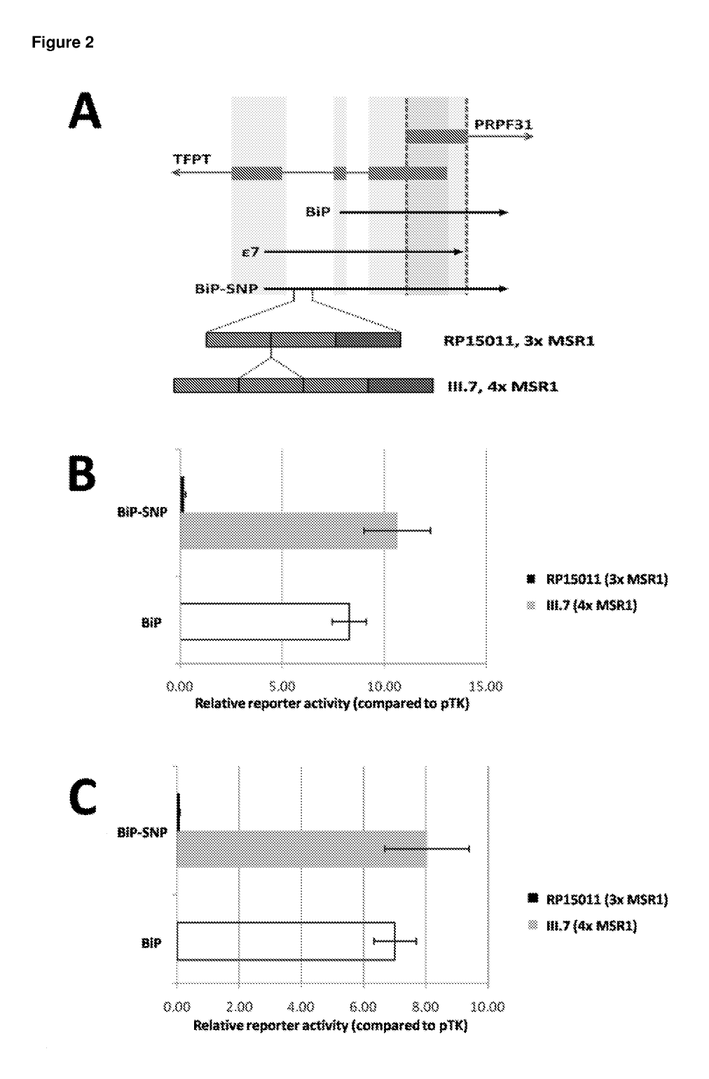

The present work shows the full effect of CNV of MSR1 element is seen when tested in the natural relation to the PRPF31 core promoter. A luciferase reporter construct was made that contained the full PRPF31 core promoter and the upstream region, including the MSR1 element in both copy number variants (FIG. 2A). Here, it was demonstrated that CNV had a dramatic effect on reporter activity, with a 53 to 115-fold higher expression in the 4-copy construct than the 3-copy construct (FIG. 2B-C) Furthermore, CNV of an MSR1 element cluster located within the 3′ untranslated region of KLK14 was shown to have a significant effect on luciferase reporter activity.

Given the large functional effect of MSR1 CNV on gene expression and the potential for an important regulatory element in the human genome a genome wide search was performed for MSR elements, identifying 978 MSR1 clusters across the genome. Clusters are predominantly located on chromosome 19 (557/978 clusters) and the sequence is most highly conserved on this chromosome. There are, however, divergent MSR1 sequences located on all human chromosomes, except the mitochondrial genome (Tables 1 and 2).

Beyond chromosome 19 the sequence is less frequent and less well conserved but it is anticipated that these elements could have regulatory potential and therefore, influence susceptibility to other biological conditions especially cancers and autoimmune diseases through differential expression of cancer-associated and immunity—associated genes.

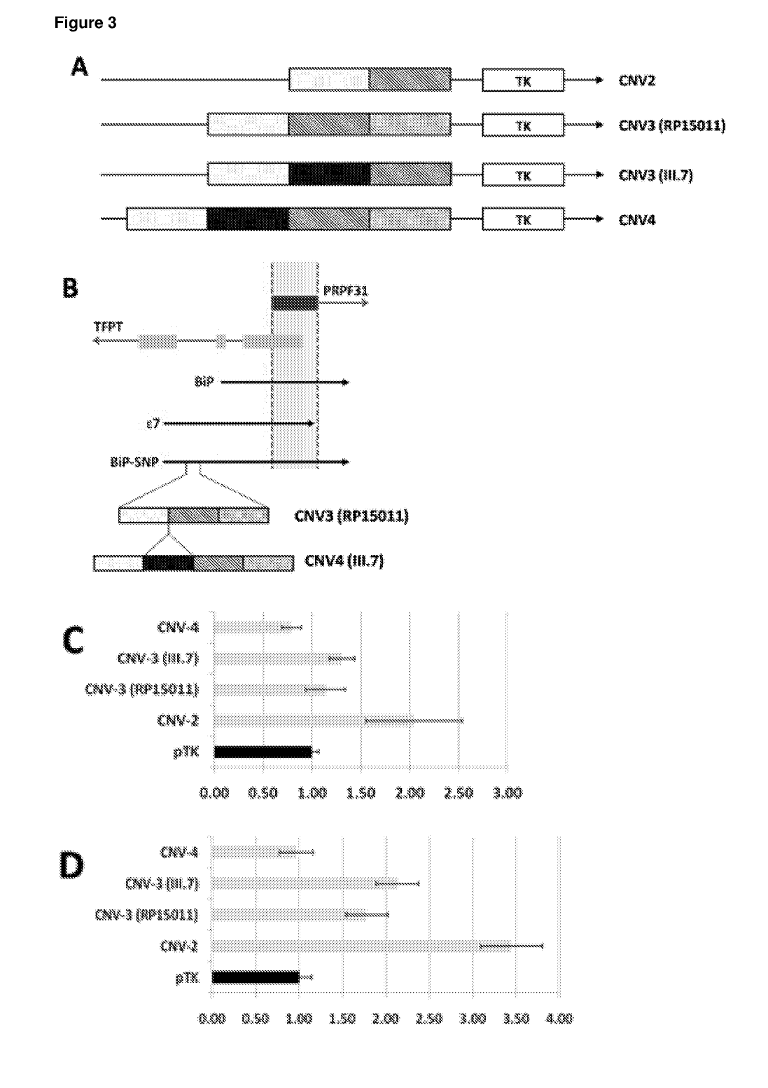

To further explore the role of MSR1 elements in gene regulation, the PRPF31 MSR1 cluster was cloned upstream to pTK minimal promoter in a pGL3 vector, at variable copy numbers (FIG. 3A-B). It was demonstrated that increasing copy number of MSR1 resulted in a significant decrease in luciferase reporter activity in HeLa cell line (FIG. 3C-D). This effect was independent of MSR1 minor sequence differences between 111.7 and RP15011 in the 3-copy constructs. In the positive strand orientation, the 2-copy construct had 2.1-fold higher activity than the 3-copy constructs; the 4-copy construct had 2.4-fold lower activity than the 3-copy constructs. In the negative strand orientation, the 2-copy construct had 1.7-fold higher activity than the 3-copy constructs; the 4-copy construct had 1.5-fold lower activity than the 3-copy constructs. This situation mimics that observed in a previous study, where the 3-copy MSR1 (in isolation) had moderately increased activity compared to the 4-copy element [8]. It is, however, in contrast to the results reported in FIG. 2 where the 4-copy element had markedly increased activity compared to the 3-copy element. This indicates that the effect of MSR1 CNV is dependent on the spatial relation of the repeat elements to the promoter region.

According to the present invention, it is predicted that when the 2-, 3-, or 4-copies alleles are cloned into pTK promoter with a filler sequence (approximately 180 bp between the MSR1 elements and TK sequence, mimicking the natural relation to PRPF31 promoter), the same dramatic effect will be seen.

It is also predicted that alteration of the distance between the MSR1 elements and the promoter sequence will allow step-wise titration of gene expression.

### Diagnostic Uses of Copy Number Variation of MSR1 Clusters

According to the present invention, copy number variation (CNV) of MSR1 clusters has been shown to be the major factor responsible for autosomal dominant retinitis pigmentosa associated with mutations in PRPF31.

CNV at several loci has been implicated in risk of developing cancer. It is highly likely, therefore, that analysis of MSR1 clusters at other loci will reveal links between CNV and both Mendelian and polygenic disorders.

It is predicted that tests that use the assessment of MSR1 CNV will have profound implications for diagnosis and prediction of human diseases. A genome wide list of MSR1 clusters frequency is seen in Table 2, and genotyping of any MSR element or derivative sequences could be used as a diagnostic or predictive test.

The work described herein demonstrates for the first time that copy number variation (CNV) of MSR1 elements has a functional effect on gene expression at two loci (PRPF31 and KLK14) and suggests a global mechanism underlying susceptibility and correlation of CNV of MSR1 elements and disease pathogenesis.

For example, previously diagnostic testing could only determine whether an individual is a PRPF31 mutation carrier. Through identification of the first molecular factor underlying the symptomatic trait—CNV of the PRPF31 MSR1—the present invention now provides means for a diagnostic test that can predict asymptomatic mutation carriers with a higher degree of certainty. By enabling a determination of whether that individual will be symptomatic or asymptomatic the present invention enables a more informed genetic counselling in PRPF31-associated adRP.

In one aspect of the present invention there is provided a test for assessing CNV of the MSR1 element at PRPF31 locus. Simple tests for defining genotype would include PCR based systems, for example using fluorescently labelled primers and sizing the PCR product against a standard ladder.

A search for other cancer genes associated with MSR1 elements on Chromosome 19 has been carried out and the results are now shown [See Table 3]. According to the present invention CNV at any of these loci could be useful in diagnostic testing to identify those individuals at risk of the associated cancers.

### Demonstration of Functional Effect of CNV of MSR1 Elements on Gene Expression PRPF31 Loci

To investigate the potential role of this functional polymorphism, the MSR1 element was studied in the context of BiP (the core promoter of PRPF31). A fragment was designed that encompassed the MSR1 polymorphism and the full PRPF31 core promoter, this fragment being termed BiP-SNP (FIG. 2A). BiP-SNP was amplified using DNA from a symptomatic individual, RP15011, harbouring the reference sequence (3×MSR1 repeat) and an asymptomatic individual, 111.7, carrying the duplication (4×MSR1 repeat). The fragment was cloned into a luciferase reporter vector and assayed by dual-luciferase reporter assay (as described in [8]).

In both cell lines tested, the fragment containing 4 copies of MSR1 had strong reporter activity [10.63±1.63 (HeLa); 8.05±1.36 (RPE-1)] (FIGS. 2B and 2C). This activity was higher than the original fragment, Bi-P, which had approximately 8-fold induction over pTK. The increased activity of 4-copy MSR1 BiP-SNP over Bi-P was significant in HeLa cell line (Mann-Whitney U=127, p<0.001), but not significant in RPE-1 cell line (Mann-Whitney U=123, p=0.056).

Strikingly, BiP-SNP containing 3 MSR1 repeats had no luciferase reporter activity [0.20±0.07 (HeLa); 0.07±0.03 (RPE-1)] (FIGS. 2B and 2C). This result is in contrast to the previously-reported results, where increased copy number of MSR1 decreased luciferase reporter activity [8]. In this assay, it was observed that increased copy number of the MSR1 element, in the natural context of the PRPF31 core promoter, massively increased luciferase reporter activity. In HeLa cell line, the construct containing 4 MSR1 repeats had 53 times higher reporter activity than the construct containing 3 MSR1 repeats, this difference was statistically significant (two-tailed Mann-Whitney U=168, p<2×10⁻⁷). An even more greater difference in activity was observed in RPE-1 cell line, where the construct containing 4 MSR1 repeats had 115 times higher activity, this also being significant (two-tailed Mann-Whitney U=306, p<4×10⁻¹⁰).

In light of the large functional effect of MSR1 CNV, the genotypes of 45 symptomatic and 28 asymptomatic individuals harbouring PRPF31 mutations were sought. The 4-copy allele was present in the heterozygous (or hemizygous) state in 7/28 asymptomatic individuals (25%), this proportion being similar to that found in the general population. It was determined that only 2/45 symptomatic individuals carried the 4-copy allele in the heterozygous state (4.5%), this being significantly under-represented compared to the general population (z=4.969, p<6.7×10⁻⁷). This means that inheritance of a 4-copy allele is highly protective against clinical manifestation of PRPF31 mutations.

### Sensitivity, Specificity and Predictive Value of Test

A test model was evaluated, with a positive result being regarded as having only 3-copy alleles (homozygous or hemizygous) and a negative result being regarded as possessing any 4-copy allele (homozygous, heterozygous or hemizygous).

| | Symptomatic | Asymptomatic |
|---|---|---|
| Positive (3-copies) | 43 (true positives) | 21 (false positives) |
| Negative (4-copies) | 2 (false negatives) | 7 (true negatives) |

The test was found to have a high sensitivity, meaning that in there is a high probability that the test result will be positive in symptomatic individuals (95.56%; 95% confidence interval (CI)=84.82-99.33%). There was, however, poor specificity, meaning that there is a low probability that the test will be negative in asymptomatic individuals (25%; 95% CI=10.74-44.88%). Overall, the positive predictive value of the test was estimated at 67%, meaning that there is a 67% chance that an individual with only 3-copy alleles will be symptomatic (67.19%; 95% CI=54.31-78.42%). Most usefully, the negative predictive value of the test was estimated at 78% (77.78%; 95% CI=40.06-96.53%), meaning that if a negative result was found (4-copy allele), it could be stated with 78% certainty that that individual is asymptomatic.

### KLK 14 Locus

A cluster of MSR1 repeat elements has been identified located within the 3′UTR of the KLK14 gene, which shows CNV within the normal population.

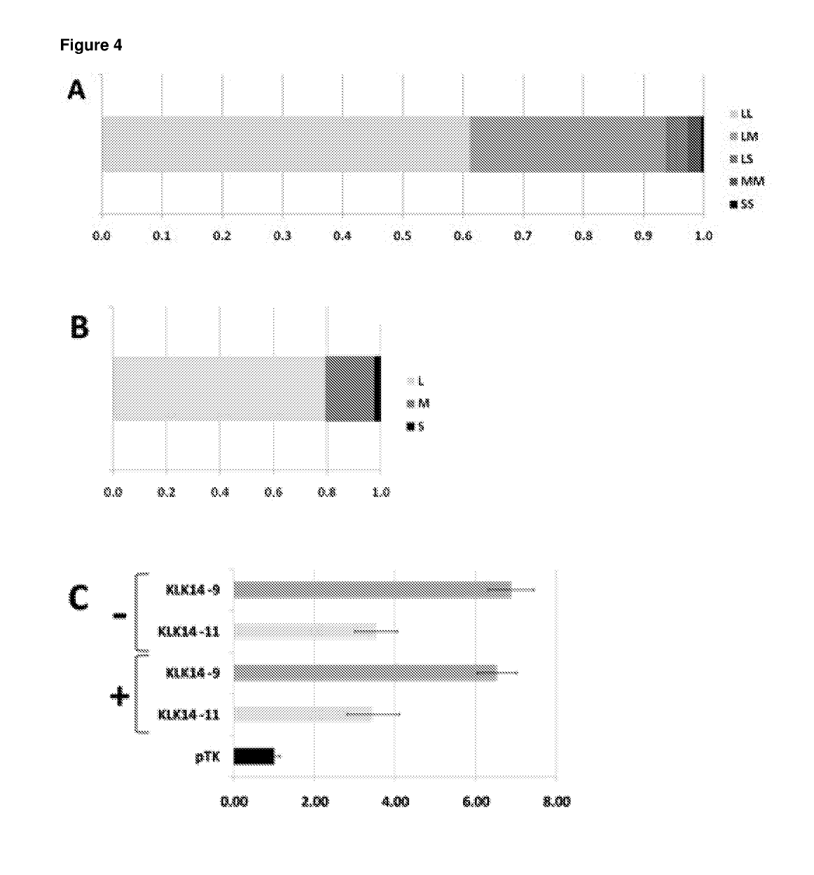

The relative frequency of alleles was assayed in 180 control individuals of European origin. The reference sequence has 11 copies and represented the majority of alleles observed (79%), but a 9-copy allele was also prevalent (18% alleles); a rare 8-copy allele also exists (2% alleles) (FIG. 4A-B).

It has been reported that the 9-copy allele was significantly more frequent in a small sample of patients with histologically confirmed breast cancer compared to matched control individuals [3]. Furthermore, KLK14 expression is dysregulated in several cancers, including breast, ovarian, prostate and testicular tumours [23].

The two alleles were cloned into pTK vector in both forward and reverse strand orientation and reporter activity detected by dual luciferase reporter assay in RPE-1 cell line. It was demonstrated that the 9-copy allele had significantly higher reporter activity in both orientations (FIG. 4C). It is predicted that this result will be reproducible in a second cell line (HeLa cells) and any other cell line tested.

### Agonism and Antagonism of MSR1 Sequences

A drug that alters the activity of MSR1 elements directly or indirectly could provide powerful therapy/treatment for many diseases, as it has the potential to alter gene expression of any of the genes that are naturally controlled by MSR1 clusters.

An agonistic (activating) compound could be used to upregulate expression of a gene or genes. Agonism of MSR1 clusters would be particularly useful for autosomal dominant conditions associated with mutations in MSR1 containing genes, where the disease mechanism is haploinsufficiency or loss of function. It is possible that there are many polygenic diseases where agonism of MSR1 elements would be of therapeutic value (Table 3 and 4).

An antagonistic (deactivating) compound could be used to downregulate expression of a gene or genes. This would be an extremely useful therapeutic approach in diseases where over-expression of MSR1 containing genes is important to pathogenesis, such as many types of cancer. It is possible that there are many polygenic diseases where antagonism of MSR1 elements would be of therapeutic value (Table 3 and 4)

Pharmaceutical manipulation of any of these sequences could be used as a therapeutic approach. It is likely to be necessary that tissue-specific and MSR1-specific drugs will need to be developed, to circumvent the problem of simultaneous dysregulation of gene expression at many loci. A genome wide list of MSR element cluster-frequencies is seen in Table 1.

### Gene Therapy Approach

MSR1 elements could be used as a powerful therapeutic tool in the context of gene therapy.

Gene therapy is the field of medicine concerned with the treatment and cure of disease through the administration of genetic material to a patient. The most commonly used gene therapy strategy involves the use of viral vectors. This system uses natural-occurring viruses that have been modified to be non-pathogenic as a “vehicle” for gene delivery, examples of commonly used viral vectors include retroviruses, adenovirus and adeno-associated virus (AAV). The AAV group are the most commonly used viral vector, as they have proven to be the safest, most efficient and provide good long-term stable gene transfer. Non-viral gene vectors are also sometimes used, which consist of a DNA plasmid delivered by a non-viral “vehicle”. Examples include chemical carriers (such as cationic polymers, lipids, detergents, and peptide-based technologies) or nanoparticles.

One critical aspect of gene therapy is the ability to control the level of expression of the transduced gene, so sufficient level is achieved for cure, without potentially harmful gene over-expression. This concept is relevant to all gene therapy, regardless of the vector system used. The lack of robust methods to regulate level of transduced gene expression has hindered progress in the gene therapy field. Clinical trials of gene therapy—such as in Parkinson's disease [26]—have been carried out in patients with late-stage or end-stage disease because of safety concerns prohibiting earlier intervention, leading to disappointing results [27]. The development of vectors with tight gene expression control has, therefore, become an important focus in the field of gene therapy.

The generic MSR1 sequence could be used to control the expression of the required gene in vector delivery systems, such as AAV, other viral vectors and non-viral systems. All vectors contain a core promoter sequence that drives the expression of the required gene. It has been demonstrated that, when located adjacent to the core promoter, variable copy number of MSR1 can alter gene expression (FIG. 3).

In this case, 2 copies of the element enhanced reporter activity (over pTK core promoter alone) by approximately 2.8-fold, 3 copies of the element modestly increased activity (1.3-fold), whereas 4 copies of the element decreased reporter activity (0.6-fold). It can be seen that this was a step-wise effect, and so it is possible that higher copies of MSR1 element would continue to decrease reporter activity, whereas one copy might increase activity further. In this example, it is clear that this strategy would allow careful control over gene expression of a transduced gene.

Furthermore, it is predicted that more dramatic changes in gene expression could be mediated by introduction of a “filler sequence” between the MSR1 element and the core promoter.

It was shown that MSR1 CNV had a dramatic effect on reporter activity when the repeat elements were separated from the core promoter by approximately 180 bp (FIG. 2). It is likely that alteration of this sequence length, in combination with alterations in MSR1 copy number and minor alterations to sequence, would produce varying effects on gene expression level. This strategy could, therefore, allow for development of “gene titration”, where the level of gene expression in a vector could be tightly controlled, until optimal gene expression was achieved.

It would be possible to apply this process in in vitro development of gene therapies to determine the correct level of gene expression, as well as in clinical trial (in vivo), where an initial dose of the gene could be used and altered according to clinical result.

### REFERENCES

1. Das H K, Jackson C L, Miller D A, et al. (1987) The human apolipoprotein C-II gene sequence contains a novel chromosome 19-specific minisatellite in its third intron. J Biol Chem.; 262(10):4787-93.
2. Jurka J, Walichiewicz J, Milosavljevic A. (1992) Prototypic sequences for human repetitive DNA. J Mol Evol.; 35(4):286-91
3. Yousef G M, Bharaj B S, Yu H, et al. (2001) Sequence analysis of the human kallikrein gene locus identifies a unique polymorphic minisatellite element. Biochem Biophys Res Commun.; 285(5):1321-9.
4. Hooper J D, Bui L T, Rae F K, et al. (2001) Identification and characterization of KLK14, a novel kallikrein serine protease gene located on human chromosome 19q13.4 and expressed in prostate and skeletal muscle. Genomics.; 73(1):117-22.
5. Nelson P S, Gan L, Ferguson C, et al. (1999) Molecular cloning and characterization of prostase, an androgen-regulated serine protease with prostate-restricted expression. Proc Natl Acad Sci USA.; 96(6):3114-9.
6. Yoshida S, Taniguchi M, Hirata A, et al. (1998) Sequence analysis and expression of human neuropsin cDNA and gene. Gene.; 213(1-2):9-16.
7. Bhaysar P K, Brand N J, Yacoub M H, et al. (1996) Isolation and characterization of the human cardiac troponin I gene (TNNI3). Genomics.; 35(1):11-23.
8. Rose A M, Shah A Z, Waseem N H, et al. (2012) Expression of PRPF31 and TFPT: regulation in health and retinal disease. Hum Mol Genet.; 21(18):4126-37.
9. Hu J C, Zhang C, Sun X, et al. (2000) Characterization of the mouse and human PRSS17 genes, their relationship to other serine proteases, and the expression of PRSS17 in developing mouse incisors. Gene; 251(1):1-8.
10. Lai J, Lehman M L, Dinger M E, et al. (2010) A variant of the KLK4 gene is expressed as a cis sense-antisense chimeric transcript in prostate cancer cells. RNA.; 16(6):1156-66.
11. Al-Maghtheh M, Vithana E, Tarttelin E, et al. (1996) Evidence for a major retinitis pigmentosa locus on 19q13.4 (RP11) and association with a unique bimodal expressivity phenotype. Am J Hum Genet.; 59(4):864-71.
12. Vithana E N, Abu-Safieh L, Allen M J, et al. (2001) A human homolog of yeast pre-mRNA splicing gene, PRP31, underlies autosomal dominant retinitis pigmentosa on chromosome 19q13.4 (RP11). Mol Cell.; 8(2):375-81.
13. Waseem N H, Vaclavik V, Webster A, et al. (2007) Mutations in the gene coding for the pre-mRNA splicing factor, PRPF31, in patients with autosomal dominant retinitis pigmentosa. Invest Ophthalmol Vis Sci.; 48(3):1330-4.
14. Vithana E, Al-Maghtheh M, Bhattacharya S S, et al. (1998) RP11 is the second most common locus for dominant retinitis pigmentosa. J Med Genet.; 35(2):174-5.
15. Vithana E N, Abu-Safieh L, Pelosini L, et al. (2003) Expression of PRPF31 mRNA in patients with autosomal dominant retinitis pigmentosa: a molecular clue for incomplete penetrance? Invest Ophthalmol Vis Sci.; 44(10):4204-9.
16. Rio Frio T, Civic N, Ransijn A, et al. (2008) Two trans-acting eQTLs modulate the penetrance of PRPF31 mutations. Hum Mol Genet.; 17(20):3154-65.
17. McGee T L, Devoto M, Ott J, et al. (1997) Evidence that the penetrance of mutations at the RP11 locus causing dominant retinitis pigmentosa is influenced by a gene linked to the homologous RP11 allele. Am J Hum Genet.; 61(5):1059-66.
18. Rose A M, Shah A Z, Venturini G, et al. (2013) Dominant PRPF31 Mutations Are Hypostatic to a Recessive CNOT3 Polymorphism in Retinitis Pigmentosa: A Novel Phenomenon of “Linked Trans-Acting Epistasis”. Ann Hum Genet. doi: 10.1111/ahg.12042.
19. Goode E L, Chenevix-Trench G, Song H, et al. (2010) A genome-wide association study identifies susceptibility loci for ovarian cancer at 2q31 and 8q24. Nat Genet.; 42(10):874-9.
20. Peedicayil A, Vierkant R A, Hartmann L C, et al. (2010) Risk of ovarian cancer and inherited variants in relapse-associated genes. PLoS One.; 5(1):e8884.
21. Hartmann L C, Lu K H, Linette G P, et al. (2005) Gene expression profiles predict early relapse in ovarian cancer after platinum-paclitaxel chemotherapy. Clin Cancer Res.; 11(6):2149-55.
22. Yousef G M, Magklara A, Chang A, et al. (2001) Cloning of a new member of the human kallikrein gene family, KLK14, which is down-regulated in different malignancies. Cancer Res.; 61(8):3425-31.
23. Borgono C A, Diamandis E P (2004) The emerging roles of human tissue kallikreins in cancer. Nat Rev Cancer. 4:876-890.
24. Adusumilli P S, Chan M K, Chun Y S, et al. (2006) Cisplatin-induced GADD34 upregulation potentiates oncolytic viral therapy in the treatment of malignant pleural mesothelioma. Cancer Biol Ther.; 5(1):48-53.
25. Foekens J A, Diamandis E P, Yu H, et al. (1999) Expression of prostate-specific antigen (PSA) correlates with poor response to tamoxifen therapy in recurrent breast cancer. Br J Cancer.; 79 (5-6):888-94.
26. Marks W J Jr, Bartus R T, Siffert J, et al. (2008) Gene delivery of AAV2-neurturin for Parkinson's disease: a double-blind, randomised, controlled trial. Lancet Neurol.; 9(12):1164-72.
27. Manfredsson F P, Bloom D C, Mandel R J. (2012) Regulated protein expression for in vivo gene therapy for neurological disorders: progress, strategies, and issues. Neurobiol Dis.; 48(2):212-21.
28. Stegh A H, Kim H, Bachoo R M, et al. (2007) BcI2L12 inhibits post-mitochondrial apoptosis signaling in glioblastoma. Genes Dev.; 21(1):98-111.
29. McKeithan T W, Takimoto G S, Ohno H, et al. (1997) BCL3 rearrangements and t(14; 19) in chronic lymphocytic leukemia and other B-cell malignancies: a molecular and cytogenetic study. Genes Chromosomes Cancer.; 20(1):64-72.
30. Hishiki T, Ohshima T, Ego T, et al. (2007) BCL3 acts as a negative regulator of transcription from the human T-cell leukemia virus type 1 long terminal repeat through interactions with TORC3. J Biol Chem.; 282(39):28335-43.
31. Eeles R A, Kote-Jarai Z, Giles G G, et al. (2008) Multiple newly identified loci associated with prostate cancer susceptibility. Nat Genet.; 40(3):316-21.
32. Ahn J, Berndt S I, Wacholder S, et al. (2008) Variation in KLK genes, prostate-specific antigen and risk of prostate cancer. Nat Genet.; 40(9):1032-4
33. Stamey T A, Yang N, Hay A R, et al. (1987) Prostate-specific antigen as a serum marker for adenocarcinoma of the prostate. N Engl J Med.; 317(15):909-16.
34. Lose F, Srinivasan S, O'Mara T, et al. (2012) Genetic association of the KLK4 locus with risk of prostate cancer. PLoS One.; 7(9):e44520.
35. Talieri M, Zoma M, Devetzi M, et al. (2012) Kallikrein-related peptidase 6 (KLK6) gene expression in intracranial tumors. Tumour Biol.; 33(5):1375-83.
36. Rechreche H, Mallo G V, Montalto G, et al. (1997) Cloning and expression of the mRNA of human galectin-4, an S-type lectin down-regulated in colorectal cancer.; 248(1):225-30.
37. Suh Y S, Lee H J, Jung E J, et al. (2012) The combined expression of metaplasia biomarkers predicts the prognosis of gastric cancer. Ann Surg Oncol.; 19(4):1240-9.
38. Barrow H, Guo X, Wandall H H, et al. (2011) Serum galectin-2, -4, and -8 are greatly increased in colon and breast cancer patients and promote cancer cell adhesion to blood vascular endothelium. Clin Cancer Res.; 17(22):7035-46.
39. Tripodi D, Quéméner S, Renaudin K, et al. (2009) Gene expression profiling in sinonasal adenocarcinoma. BMC Med Genomics.; 2:65.
40. Fukunaga-Johnson N, Lee S W, Liebert M, et al. (1996) Molecular analysis of a gene, B B1, overexpressed in bladder and breast carcinoma. Anticancer Res.; 16(3A):1085-90.
41. Korabiowska M, Betke H, Kellner S, et al. (1997) Differential expression of growth arrest, DNA damage genes and tumour suppressor gene p53 in naevi and malignant melanomas. Anticancer Res.; 17(5A):3697-700.
42. Seo Y, Matozaki T, Tsuda M, et al. (1997) Overexpression of SAP-1, a transmembrane-type protein tyrosine phosphatase, in human colorectal cancers. Biochem Biophys Res Commun.; 231(3):705-11.
43. Matozaki T, Suzuki T, Uchida T, et al. (1994) Molecular cloning of a human transmembrane-type protein tyrosine phosphatase and its expression in gastrointestinal cancers. J Biol Chem.; 269(3):2075-81.

TABLE 1

| Chromosome | Occurrences |
|---|---|
| 1 | 44 |
| 2 | 30 |
| 3 | 10 |
| 4 | 4 |
| 5 | 8 |
| 6 | 22 |
| 7 | 110 |
| 8 | 12 |
| 9 | 19 |
| 10 | 21 |
| 11 | 13 |
| 12 | 10 |
| 13 | 13 |
| 14 | 11 |
| 15 | 4 |
| 16 | 26 |
| 17 | 26 |
| 18 | 12 |
| 19 | 557 |
| 20 | 4 |
| 21 | 6 |
| 22 | 3 |
| Mitochondrial | 0 |
| X | 9 |
| Y | 4 |

TABLE 2

| Chromosome | Gene | Chromosome | Gene |
|---|---|---|---|
| 5 | CLPTM1L | 7 | ADAP1 |
| | SLC12A7 | | FBXL18 |
| | SLC6A19 | | FOXK1 |
| | TERT | | GET4 |
| | TPPP | | SUN1 |
| | ZDHHC11 | | TNRC18 |
| | | | RASA4 |
| | | | UPK3BL |
| X | CRLF2 | 16 | IL17C |
| | PLCXD1 | | PRDM7 |
| 14 | ASB2 | | RPL13 |
| | CPNE6 | | SPG7 |
| | NDRG2 | | TCF25 |
| | NFATC4 | | TUBB3 |
| 8 | FLJ43860 | | GPT2 |
| | FOXH1 | | AK128777 |
| | GML | | CLDN9 |
| | SCXB | | CRAMP1L |
| | LY6E | | HN1L |
| | LY6H | | MMP25 |
| | TSNARE1 | | PKMYT1 |
| 6 | BTNL2 | | RAB11FIP3 |
| | BAK1 | | RAB40C |
| | CYP21A2 | | SOLH |
| | HLA cluster genes | | SOX8 |
| | NOTCH4 | | SSTR5 |
| | SMOC2 | | EIF3C |
| | TNXB | | |
| | VEGFA | | |
| 17 | C17orf57 | 1 | TP73 |
| | EZH1 | | MDM4 |
| | RASD1 | | FBXO2 |
| | STAT3 | | GRIK3 |
| | TIMM22 | | H6PD |
| | BTBD17 | | IPO9 |
| | HLF | | MPZL1 |
| | KIF19 | | WNT9A |
| | SLC39A11 | | |
| | SOX9 | | |
| | STXBP4 | | |
| | TIMP2 | | |
| | USP36 | | |
| 2 | INHBB | | |
| | SIX2 | | |
| | GLI2 | | |
| | ID2 | | |
| | OSR1 | | |
| | SIX2 | | |
| | SNED1 | | |
| 19 | APLP1 | | |
| | APOE | | |
| | CAPN12 | | |
| | CD177 | | |
| | FCGRT | | |
| | FGF21 | | |
| | HSD17B14 | | |
| | IGLON5 | | |
| | IRF3 | | |
| | KDELR1 | | |
| | KLK6 | | |
| | KLK8 | | |
| | LYPD5 | | |
| | MAMSTR | | |
| | MYBPC2 | | |
| | NCCRP1 | | |
| | NKG7 | | |
| | NTF4 | | |
| | PLEKHA4 | | |
| | PRPF31 | | |
| | RDH13 | | |
| | RRAS | | |
| | SBK2 | | |
| | SCAF1 | | |
| | TEAD2 | | |
| | TNNI3 | | |
| | CACNG6 | | |
| | CNFN | | |
| | DKKL1 | | |

Table 2 Notes:
1. Chromosome 6 genes are associated with a number of different diseases, including: systemic lupus erythaematosus, mucocutaneous lymph node syndrome, lymphadenitis, purpura, sarcoidosis, polyarteritis nodosa, vascular disease, vasculitis, Behcet's disease, congenital adrenal hyperplasia, psoriasis, renal cell carcinoma
2. A small selection of chromosome 19 genes where the MSR1 is in putative promoter, therefore highly likely to be regulated by MSR1. However, as demonstrated at KLK14 locus MSR1 elements outside of the promoter can have a functional effect.
3. On other chromosomes (other than 19) the genes were selected by coincidence of an MSR1 element cluster and reported regulatory function of the genomic region (according to ENCODE project).

TABLE 3

| | | | | Refer- |
|---|---|---|---|---|
| Gene | Start | End | Associated cancer(s) | ence |
| BCL2L12 | 50169615 | 50169761 | Glioblastoma | [28] |
| | 50169789 | 50169899 | | |
| BCL3 | 45259604 | 45259986 | B-cell leukaemia and | [29, 30] |
| | 45262119 | 45262257 | lymphoma | |
| | 45262320 | 45262429 | Chronic lymphocytic | |
| | 45262467 | 45262665 | leukaemia | |
| KLK3 | 51361577 | 51361661 | Prostate | [3, |
| | | | Ovarian | 31-32] |
| | | | Breast | |
| KLK4 | 51409713 | 51410118 | Ovarian | [3, 23, |
| | | | Prostate | 33-34] |
| KLK6 | 51472206 | 51472500 | Glioblastoma and other | [23, 35] |
| | | | intracranial tumours | |
| | | | Breast | |
| | | | Ovarian | |
| | | | Prostate | |
| | | | Colon | |
| | | | Pancreatic | |
| KLK7 | 51483235 | 51483487 | Breast | [23] |
| | 51485737 | 51486917 | Ovarian | |
| KLK8 | 51502449 | 51502548 | Breast | [23] |
| | 51503557 | 51503648 | Cervical | |
| | 51504549 | 51504729 | Colon | |
| | | | Ovarian | |
| KLK9 | 51506581 | 51506671 | Breast | [23] |
| | 51506703 | 51506844 | Ovarian | |
| KLK14 | 51580818 | 51581230 | Breast | [23] |
| | 51582243 | 51582696 | Ovarian | |
| | | | Prostate | |
| | | | Testicular | |
| LGALS4 | 39292836 | 39292933 | Colorectal | [36-39] |
| | | | Gastric | |
| | | | Breast | |
| | | | Sinonasal | |
| | | | adenocarcinoma | |
| MBOAT7 | 54678157 | 54678496 | Breast | [40] |
| | | | Bladder | |
| PPP1R15A | 49375942 | 49376023 | Melanoma | [41] |
| | 49376218 | 49376307 | | |
| PRPF31 | 54618105 | 54618472 | Ovarian | [20] |
| PTPRH | 55718905 | 55720407 | Colorectal | [42-43] |
| | 55720623 | 55720713 | Gastric | |
| | | | Pancreatic | |
| KLK10 | 51518890 | 51519105 | Acute Lymphoblastic | [23] |
| | | | Leukaemia | |
| | | | Breast | |
| | | | Colon | |
| | | | Ovary | |
| | | | Squamous Cell | |
| | | | Carcinoma | |
| | | | Pancreas | |
| | | | Prostate | |
| | | | Testicular | |

TABLE 4
Other chromosome 19 MSR1 clusters, where the
gene has a significant disease association

| | MSR1 position(s) hg19 co- | | |
|---|---|---|---|
| | ordinates | | |
| Gene | Start | End | Associated diseases |
| APLP1 | 36369600 | 36369716 | Alzheimer's disease |
| APOC4 | 45448179 | 45448284 | Hypercholesterolaemia |
| | 45452173 | 45452396 | |
| APOE | 45408659 | 45408722 | Alzheimer's disease |
| | | | Hypercholesterolaemia |
| | | | Dyslipidaemia |
| | | | Cardiovascular disease |
| CA11 | 49142917 | 49143027 | Bipolar disease |
| | 49147902 | 49148001 | |
| | 49148530 | 49148643 | |
| CD209 | 7811596 | 7811693 | Susceptibility to HIV, TB, |
| | | | leprosy, Dengue fever |
| | 7811850 | 7812170 | |
| DKKL1 | 49865844 | 49866063 | Multiple sclerosis |
| FGF21 | 49259006 | 49259154 | Bipolar disease |
| FUT1 | 49259006 | 49259154 | Obesity |
| | | | Metabolic syndrome |
| GYS1 | 49485843 | 49485960 | Type 2 Diabetes |
| | 49488901 | 49489028 | Hypertension |
| | 49494939 | 49495074 | Obesity |
| LIM2 | 51890804 | 51891165 | Cataract |
| NTF4 | 49561687 | 49561816 | Glaucoma |
| | 49567587 | 49568025 | |
| SLC17A7 | 49937404 | 49937856 | Nicotine dependence |
| | 49940190 | 49940283 | |
| TOMM40 | 45404109 | 45404218 | Alzheimer's disease |
| | | | Metabolic syndrome |
| | | | Cardiovascular disease |

1. The use of minisatellite repeat element 1 (MSR1) in a process of identifying therapeutic agents for use in the treatment or therapy of diseases or conditions relating to one or more genes associated a minisatellite repeat element 1 (MSR1) or functional variants or derivatives thereof.

2. (canceled)

3. The use according to claim 1 wherein therapeutic agents are designed or identified which can alter the activity of MSR1 elements directly or indirectly or bind to MSR1 to either activate (agonise) or deactivate (antagonise) the MSR1 sequence.

4. A test for the prediction, diagnosis, prognosis or response to therapy in a disease or condition in a subject, said disease or condition relating to one or more genes associated with a minisatellite repeat element 1 (MSR1) or functional variants or derivatives thereof wherein said test comprises a means for assessing the copy number variation (CNV) at an MSR1 locus of the gene or genes so as to determine the risk of the disease or condition being present or developing in the subject.

5. The test according to claim 4 wherein the gene is selected from one or more genes shown in Tables 2 or 3.

6. The test according to claim 4 wherein the gene is selected from one or more cancer genes or PRPF31.

7. The test according to claim 6 wherein the cancer is selected from those listed in Table 3.

8. The test according to claim 4 wherein the gene is KLK4 or KLK14.

9. The test according to claim 4 which is PCR based.

10. (canceled)

11. (canceled)

12. (canceled)

13. The test according to claim 4 wherein the gene is PRPF31 and the disease or condition is ovarian cancer or autosomal dominant retinitis pigmentosa.

14. The test according to claim 4 wherein the gene is KLK14 and the disease or condition is breast cancer.

15. The test according to claim 4 wherein the gene is KLK4 and the disease or condition is prostate cancer.

16. A test for predicting the risk of PRPF31-associated autosomal dominant retinitis pigmentosa (adRP), the test comprising assessing the copy number variation (CNV) of the MSR1 element at the PRPF31 locus so as to determine the risk of PRPF31-associated adRP being present or developing in the subject; wherein a CNV of three indicates an increased risk of PRPF31-associated adRP and a CNV of four indicates a decreased risk of PRPF31-associated adPR.

17. The test according to claim 16 which is PCR based using fluorescently labelled primers and sizing the PCR product against a standard ladder.
